(19) United States
(12) Patent Application Publication
(10) Pub. No.: US 2014/0011838 A1
(43) Pub. Date: Jan. 9, 2014

(54) COMPOSITIONS AND METHODS FOR TREATING WARTS ASSOCIATED WITH VIRAL INFECTIONS

(71) Applicant: Justice E. OBI, St. Albans, NY (US)

(72) Inventor: Justice E. OBI, St. Albans, NY (US)

(21) Appl. No.: 13/687,273

(22) Filed: Nov. 28, 2012

Related U.S. Application Data
(63) Continuation-in-part of application No. 13/540,749, filed on Jul. 3, 2012.

(51) Int. Cl. A61K 31/4706

(52) U.S. Cl. CPC A61K 31/4706
USPC 514/313

(57) ABSTRACT

The invention provides compositions and methods for the treatment, as well as the inhibition and prevention, of an infection of the human papillomavirus and the epithelial lesions, namely, the warts of the skin and mucosal surfaces, associated therewith. The compositions comprise a therapeutically effective amount of an active ingredient comprising at least one compound selected from the group consisting of chloroquine, hydroxychloroquine, amodiaquine, or in each case, a pharmaceutically acceptable salt thereof. The methods comprise topically administering a therapeutically and/or antivirally effective amount of such a compound to a human being in need of such treatment, although in certain of the compounds may alternatively be administered subcutaneously or transdermally. The compositions may optionally also comprise one or more pharmaceutically acceptable non-active ingredients.

### REFERENCE TO RELATED APPLICATIONS

This application is a continuation-in-part of prior co-pending application Ser. No. 13/540,749, filed Jul. 3, 2012.

### TECHNICAL FIELD

This invention relates, in general, to compositions and methods for treating and removing warts, and more specifically to the treatment and prevention of genital warts and other warts of the skin and mucosal surfaces that are associated with an infection of the human papillomavirus.

### BACKGROUND OF THE INVENTION

The human papillomavirus is a virus of the papillomavirus family that is capable of infecting human beings; these viruses most often infect keratinocytes of the skin or mucous membranes and produce epithelial lesions or growths, particularly warts. Over 120 human papillomavirus types have been identified, some of which, e.g., types 6 and 11, which can be transmitted through skin-to-skin contact, including sexual contact, and which are associated with unsightly but non-cancerous warts on the surface of the skin, including common warts (verrucae), flat warts and plantar warts (the latter are found on the sole or toes of the foot), while other types, e.g., types 16 and 18, which are also transmitted through sexual contact and are associated with unsightly and embarrassing anogenital warts (found in body cavities such as the oral cavity, the rectum and the vaginal cavity, the latter including the vulva and cervix, but also found in other, much more visible skin surfaces, such as the male genitalia). These sexually transmitted types of the human papillomavirus are also known to play a role in, and may in fact lead to, several types of cancers, including cervical, vaginal, vulvar, penile, anal, rectal and/or oropharyngeal cancers.

Warts associated with the human papillomavirus have often been treated and/or removed using one of the following compositions and/or methods: cryotherapy, which involves freezing the abnormal cells with liquid nitrogen; conization, or a cone biopsy, which removes the abnormal areas surgically; or Loop Electrosurgical Excision Procedure (LEEP), where the abnormal cells are removed with an electrical current. For warts in less sensitive areas, acids may be applied to assist in terminating the wart; canadid antigen may be injected to stimulate the immune system to fight off the wart; a topical medication called imiquimod may be applied; or no treatment at all may be used, which implies simply waiting to see if the cells can heal on their own.

However, many of these compositions and/or methods include burning or freezing away the wart (using chemicals or an electrical current), which can often be a painful process, and in any event can leave unsightly scarring. Others utilize a medication that modifies the immune response to the virus in order to treat the wart, but these often require more time to fight the virus and terminate the wart. Additionally, while these methods may be used to target and treat existing warts, they do nothing to prevent additional warts from forming in the future.

It is therefore one of the primary objects of the present invention to provide compositions and methods for treating and removing warts associated with the human papillomavirus, particularly those associated with types 6, 11, 16 and 18.

Another object of the present invention is to provide compositions and methods for treating and removing warts associated with the human papillomavirus in which the compositions are safe enough to be used topically on highly sensitive regions of the skin, such as may be found in the genital areas.

Still another object of the present invention is to provide compositions and methods for treating and removing warts associated with the human papillomavirus which can be administered to humans in a variety of forms.

Yet another object of the present invention is to provide compositions and methods for treating and removing warts associated with the human papillomavirus which provide quick relief and termination of the wart, as well as sustained relief to prevent additional warts from forming.

### SUMMARY OF THE INVENTION

The present invention provides compositions and methods for treating an infection associated with the human papillomavirus; such an infection is generally manifested in a human as epithelial lesions, particularly warts, and most particularly skin warts and anogenital warts. The compositions of the invention for treating and removing such warts comprise at least one compound selected from the group consisting of chloroquine, hydroxychloroquine and amodiaquine. More particularly, the compositions comprise either chloroquine alone or hydroxychloroquine alone or amodiaquine alone, or pharmaceutically acceptable salts thereof, as the active ingredient, or combinations of any two, or all three, of those compounds, or pharmaceutically acceptable salts thereof, as the active ingredients. Optionally, the compositions may also comprise pharmaceutically acceptable non-active ingredients, such as ethyl alcohol, lidocaine, epinephrine and/or diphenhydramine.

The methods of the invention comprise administering chloroquine to a human being in a topical form, such as in a gel or spray, or subcutaneously in an injectable form. The methods of the invention further comprise administering hydroxychloroquine to a human being in a topical form, such as in a gel or spray, or transdermally utilizing a patch. The methods of the invention also comprise administering a chloroquine/hydroxychloroquine combination, or a chloroquine/amodiaquine combination, or a hydroxchloroquine/amodiaquine combination, to a human being in a topical form, such as in a gel or spray. The methods of the invention still further comprise administering a chloroquine/hydroxychloroquine/amodiaquine combination to a human being in a topical form, such as in a gel or spray.

Thus, one aspect of the present invention generally concerns methods for the treatment of a human subject suffering from an existing human papillomavirus infection. In one embodiment of this aspect, the methods comprise administering a therapeutically effective amount of chloroquine or a pharmaceutically acceptable salt thereof to the subject. In another embodiment the methods comprise administering a therapeutically effective amount of hydroxychloroquine or a pharmaceutically acceptable salt thereof to the subject. In yet another embodiment of this aspect of the invention, the methods comprise administering a therapeutically effective amount of chloroquine or a pharmaceutically acceptable salt thereof together with a therapeutically effective amount of amodiaquine or a pharmaceutically acceptable salt thereof to the subject. In still another embodiment, the methods comprise administering a therapeutically effective amount of hydroxychloroquine or a pharmaceutically acceptable salt thereof together with a therapeutically effective amount of amodiaquine or a pharmaceutically acceptable salt thereof to the subject.

Another aspect of the invention generally concerns methods for the prevention or inhibition of the development in a human subject of an infection of the human papillomavirus. In one embodiment of this aspect, the methods comprise administering an antivirally effective amount of chloroquine or a pharmaceutically acceptable salt thereof to the subject. In another embodiment the methods comprise administering an antivirally effective amount of hydroxychloroquine or a pharmaceutically acceptable salt thereof to the subject. In yet another embodiment of this aspect of the invention, the methods comprise administering an antivirally effective amount of chloroquine or a pharmaceutically acceptable salt thereof together with an antivirally effective amount of amodiaquine or a pharmaceutically acceptable salt thereof to the subject. In still another embodiment, the methods comprise administering an antivirally effective amount of hydroxychloroquine or a pharmaceutically acceptable salt thereof together with an antivirally effective amount of amodiaquine or a pharmaceutically acceptable salt thereof to the subject.

Another aspect of the invention generally concerns pharmaceutical compositions for the treatment of a papillomavirus infection in a human host. In one embodiment of this aspect, the compositions comprise a therapeutically effective amount of chloroquine or a pharmaceutically acceptable salt thereof. In another embodiment the compositions comprise a therapeutically effective amount of hydroxychloroquine or a pharmaceutically acceptable salt thereof. In yet another embodiment of this aspect of the invention, the compositions comprise a therapeutically effective amount of chloroquine or a pharmaceutically acceptable salt thereof together with a therapeutically effective amount of amodiaquine or a pharmaceutically acceptable salt thereof. In still another embodiment, the compositions comprise a therapeutically effective amount of hydroxychloroquine or a pharmaceutically acceptable salt thereof together with a therapeutically effective amount of amodiaquine or a pharmaceutically acceptable salt thereof.

These and other aspects, features, objects and advantages of the present invention will become more apparent to those skilled in the art from the following detailed description of the presently most preferred embodiments thereof.

### DETAILED DESCRIPTION OF THE PREFERRED EMBODIMENT

The present invention resides in the discovery that the known compounds chloroquine, hydroxychloroquine and amodiaquine, or pharmaceutically acceptable salts thereof, all of which have been used previously as antimalarial agents and/or to treat disorders of the immune system, also have utility in treating infections of the human papillamovirus, and in particular, in treating and removing the warts associated with such infections, as well as in preventing the recurrence of such warts.

The molecular structures of chloroquine, hydroxychloroquine and amodiaquine are provided below, as Formula (I), Formula (II) and Formula (III), respectively.

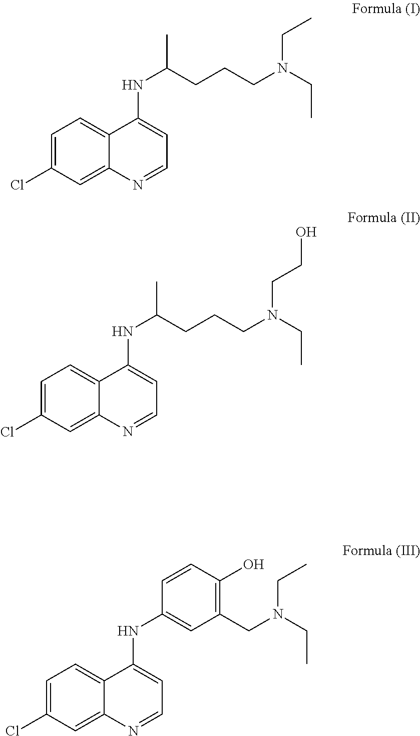 [SMILES: CCN(CC)CCCC(C)Nc1ccnc2cc(Cl)ccc12 ; CCN(CCO)CCCC(C)Nc1ccnc2cc(Cl)ccc12 ; CCN(CC)Cc1cc(Nc2ccnc3cc(Cl)ccc23)ccc1O]

As is known, and as can be seen in the structural formulas set out above, these three compounds are related to one another in that their structures have a heterodicyclic portion in common, namely, the 7-chloro derivative of the 4-aminoquinoline moiety. Chloroquine (Formula I) has been marketed under the brand name Resochin, while hydroxychloroquine (Formula II) has been marketed under the brand name Plaquenil, while amodiaquine (Formula III) has been marketed under the brand name Camoquin. Chloroquine is commercially available as the pharmaceutically acceptable salt chloroquine phosphate, and may be purchased in solid (tablet) form from a wide variety of sources such as Ohm Laboratories, Inc. of North Brunswick, N.J., USA, while a ready-made aqueous solution may be purchased from a wide variety of sources, including Sai Parenterals (P) Ltd., of Hyserabad, India, or Scott Edil Pharmacia Ltd., of Jhannajri, India. Hydroxychloroquine, in solid (tablet) form, is commercially available as the pharmaceutically acceptable salt hydroxychloroquine sulphate, and may be purchased from a wide variety of sources such as West-Ward Pharmaceutical Corporation of Eatontown, N.J., U.S.A. Amodiaquine, in solid (tablet) form, is commercially available as the pharmaceutically acceptable salt amodiaquine hydrochloride, and may be purchased from a wide variety of sources, including Parke, Davis & Company, a division of Pfizer Inc., headquartered in New York, N.Y., USA.

In the preferred embodiments, the compositions of the invention comprise either chloroquine (Formula I) alone or hydroxychloroquine (Formula II) alone or amodiaquine (Formula III) alone as the active ingredient, or either chloroquine combined with hydroxychloroquine, or chloroquine combined with amodiaquine or hydroxychloroquine combined with amodiaquine or chloroquine combined with both hydroxychloroquine and amodiaquine as the active ingredients. In general, and as set forth in further detail below, the methods of the invention comprise, in the preferred embodiments, administering the active ingredient or ingredients to a human being in a topical form, such as in a patch, gel, spray or combinations thereof, and in one case, in an injectable form.

As used herein, the term “gel” refers to a composition that contains a gelling agent and a gel-triggering agent, which when combined form a soft solid composition. Gelling agents and gel-triggering agents are generally well known in the art.

As used herein, the term “patch” refers to an adhesive material able to bind to the skin around the periphery of the area to be treated, so as to remain attached to the skin over time, and to contain a medicament in its interior. Patches are generally well known in the art.

As used herein, the term “spray” refers to a liquid mixture in droplet form that is capable of binding to the surface of the skin when applied.

As used herein, the term “injectable” refers to a solution prepared to be placed inside a syringe that will then be injected under the skin in the area to be treated.

The compositions of the invention comprising chloroquine as the only active ingredient may be administered in topical form, either as a gel or as a spray, or in a form that may be subcutaneously injected. When they are to be administered in the topical form, the compositions preferably comprise chloroquine plus optional non-active ingredients. The non-active ingredients may comprise ethyl alcohol and peppermint spirit oil, and the gel form may additionally comprise lidocaine jelly or ointment, while the spray form may additionally comprise lidocaine as a 2% solution. All of the non-active ingredients are conventional and are available commercially from a wide variety of sources. The non-active ingredients are included for the following purposes: the lidocaine functions as an analgesic, the ethyl alcohol functions as an antiseptic, while the peppermint spirit oil functions to provide a pleasing odor. A particularly preferred composition for the gel form may be prepared from the starting ingredients set forth below:

| | |
|---|---|
| Chloroquine phosphate | 4000 mg (8 500-mg tablets) |
| Lidocaine jelly 2% or ointment 5% | 10 ml or 10 mg, respectively |
| Ethyl alcohol (70% aqueous solution) | 15 ml |
| Peppermint spirit oil | 2.5 ml |
| Water | 2.5 ml |

Preferably, this composition is prepared by crushing the chloroquine phosphate tablets in a mortar, and then adding each of the non-active ingredients, in the sequence listed above, sufficient to make a total of approximately 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed lightly for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) by improving the contact between the wart and the medication. Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

When the compositions of the invention comprising cholorquine as the only active ingredient are to be administered in injectable form, the compositions preferably comprise an aqueous solution of chloroquine plus optional non-active ingredients. The non-active ingredients may comprise diphenhydramine, lidocaine solution and epinephrine. A particularly preferred composition is set forth below:

| | |
|---|---|
| Chloroquine phosphate solution | 20 ml |
| Lidocaine (2% solution) | 5 ml |
| Epinephrine Injection 1:1000 | 2.5 ml |
| Diphenhydramine 50 mg/ml | 2.5 ml |

Each milliliter of the chloroquine phosphate solution contains 64.5 mg B.P. (British Pharmacopia) of chloroquine phosphate, which is equivalent to 40 mg of chloroquine. All of the non-active ingredients are conventional and are available commercially from a wide variety of sources. The non-active ingredients are included for the following purposes: the lidocaine functions as a local anaesthetic, the diphenhydramine functions as an antihistimine to decrease any possible allergic reaction to the active ingredient, while the epinephrine functions to constrict small blood vessels. Preferably, this composition is prepared by mixing the active ingredient with the non-active ingredients (in any sequence), so as to yield a sufficient amount of the injectable form of the medication to fill a 30 ml vial.

The injectable form of this medication is preferably administered to a patient as follows. Tuberculin syringes, each having a capacity of 3 ml to 5 ml, are used to withdraw aliquots of 0.5 ml to 1 ml from the 30 ml vial. This amount is then injected (with appropriate sterilization) into or beneath the base of each wart once a day for 5-7 days, until disappearance of the wart(s). Although this injectable form is the fastest-acting form of the medication, it is the preferred form for use only on warts appearing in one or more body cavities and on the skin; it is not the preferred form for use on warts appearing on the male genitalia.

The compositions of the invention comprising hydroxychloroquine as the only active ingredient are preferably administered in topical form, as a patch, a gel or a spray. Preferably, these compositions comprise hydroxychloroquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form may be prepared from the starting ingredients set forth below:

| | |
|---|---|
| Hydroxychloroquine sulphate | 2400 mg (12 200-mg tablets) |
| Lidocaine jelly 2% or ointment 5% | 10 ml or 10 mg, respectively |
| Ethyl alcohol (70% aqueous solution) | 15 ml |
| Peppermint spirit oil | 2.5 ml |
| Water | 2.5 ml |

Preferably, this composition is prepared by crushing the hydroxychloroquine sulphate tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle. As an additional alternative, approximately 3.5 gm of the gel form may then be transferred to the pad of a conventional 3.5 mini-patch.

The gel form of this medication is preferably administered to a patient topically as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s). Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage for the spray form is preferably two puffs applied to the affected areas once or twice daily. The patch form of this medication is also preferably administered to a patient in the same manner, although the dosage for the patch form preferably constitutes applying a patch to each affected area once a day, and replacing it with a fresh patch once every 24 hours (approximately), until disappearance of the wart(s). It is to be understood that the patch form can also be used to cover the scars left after conventional surgical removal of a wart, with replacement every other day, in order to prevent re-growth of the wart.

The compositions of the invention comprising amodiaquine as the only active ingredient are preferably administered in topical form, either as a gel or as a spray. Preferably, these compositions comprise 100-200 mg of amodiaquine, plus optional non-active ingredients. The non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form is set forth below:

| | |
|---|---|
| Amodiaquine hydrochloride | 800 mg (4 200-mg tablets) |
| Lidocaine jelly 2% or ointment 5% | 10 ml or 10 gm, respectively |
| Ethyl alcohol (70% aqueous solution) | 15 ml |
| Peppermint spirit oil | 2.5 ml |
| Water | 2.5 ml |

Preferably, this composition is prepared by crushing the amodiaquine hydrochloride tablets in a mortar, and then adding each of the non-active ingredients, in the sequence listed above, sufficient to make a total of approximately 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed lightly for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) by improving the contact between the wart and the medication. Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

The compositions of the invention comprising chloroquine in combination with hydroxychloroquine as the active ingredients are preferably administered in topical form, either as a gel or as a spray. Preferably, these compositions comprise 200-500 mg of chloroquine and 150-200 mg of hydroxychloroquine, plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form is set forth below:

| | |
|---|---|
| Chloroquine phosphate | 4000 mg (8 500-mg tablets) |
| Hydroxychloroquine sulfate | 2400 mg (12 200-mg tablets) |
| Lidocaine jelly 2% or ointment 5% | 10 ml or 10 gm, respectively |
| Ethyl alcohol (70% aqueous solution) | 15 ml |
| Peppermint spirit oil | 2.5 ml |
| Water | 2.5 ml |

Preferably, this composition is prepared by crushing the chloroquine phosphate tablets together with the hydroxychloroquine sulfate tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

The compositions of the invention comprising chloroquine in combination with amodiaquine as the active ingredients are preferably administered in topical form, either as a gel or as a spray. Preferably, these compositions comprise chloroquine and amodiaquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form may be prepared from the starting ingredients set forth below:

| | |
|---|---|
| Chloroquine phosphate | 4000 mg (8 500-mg tablets) |
| Amodiaquine hydrochloride | 800 mg (4 200-mg tablets) |
| Lidocaine jelly 2% or ointment 5% | 10 ml or 10 mg, respectively |
| Ethyl alcohol (70% aqueous solution) | 15 ml |
| Peppermint spirit oil | 2.5 ml |
| Water | 2.5 ml |

Preferably, this composition is prepared by crushing the chloroquine phosphate tablets together with the amodiaquine hydrochloride tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

The compositions of the invention comprising hydroxychloroquine in combination with amodiaquine as the active ingredients are preferably administered in topical form, either as a gel or as a spray. Preferably, these compositions comprise hydroxychloroquine and amodiaquine plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition for the gel form may be prepared from the starting ingredients set forth below:

| | |
|---|---|
| Hydroxychloroquine sulphate | 2400 mg (12 200-mg tablets) |
| Amodiaquine hydrochloride | 800 mg (4 200-mg tablets) |
| Lidocaine jelly 2% or ointment 5% | 10 ml or 10 mg, respectively |
| Ethyl alcohol (70% aqueous solution) | 15 ml |
| Peppermint spirit oil | 2.5 ml |
| Water | 2.5 ml |

Preferably, this composition is prepared by crushing the hydroxychloroquine sulphate tablets together with the amodiaquine hydrochloride tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

The compositions of the invention comprising chloroquine in combination with both hydroxychloroquine and amodiaquine as the active ingredients are preferably administered in topical form, either as a gel or as a spray. Preferably, these compositions comprise 200-500 mg of chloroquine, 150-200 mg of hydroxychloroquine and 100-200 mg of amodiaquine, plus optional non-active ingredients. As above, the non-active ingredients may comprise ethyl alcohol and peppermint spirit oil, and the gel form may additionally comprise lidocaine jelly or ointment, while the spray form may additionally comprise lidocaine as a 2% solution. A particularly preferred composition is set forth below:

| | |
|---|---|
| Chloroquine phosphate | 4000 mg (8 500-mg tablets) |
| Hydroxychloroquine sulphate | 2400 mg (12 200-mg tablets) |
| Amodiaquine hydrochloride | 800 mg (4 200-mg tablets) |
| Lidocaine jelly 2% | 10 ml |
| Ethyl alcohol (70% aqueous solution) | 15 ml |
| Peppermint spirit oil | 2.5 ml |
| Water | 2.5 ml |

Preferably, this composition is prepared by crushing the chloroquine phosphate tablets together with both the hydroxychloroquine sulphate tablets and the amodiaquine hydrochloride tablets in a mortar, and then adding each of the non-active ingredients, in the sequence listed above, sufficient to make a total of about 30 gm of the gel or spray. The gel form may then be transferred to a tube or other appropriate container, while the spray form may then be transferred into a conventional spray bottle.

The gel form of this medication is preferably administered to a patient as follows. The affected areas are first cleaned with one or more alcohol swabs, and optionally, each wart to be treated may then be filed for approximately 30 seconds with a conventional nail filing device or instrument (which can be purchased from a variety of sources, including beauty supply shops); only warts that appear on the toes, fingers, elbows, knees and other hard surfaces of the patient's skin should be filed, whereas warts appearing on soft tissues, such as the genitalia or in the oral cavity, need not (and should not) be filed. This filing, although optional, is preferred since such filing accelerates removal of the wart(s) Thereafter, an amount of the gel approximately equal to the surface area of each wart (or an amount equal to the surface area of the tip of a finger) is applied to the affected area, after which that area may optionally be covered with a sterile bandage. The gel form is preferably administered to the patient in the foregoing manner once or twice a day, for approximately one to four weeks, until disappearance of the wart(s). The spray form of this medication is preferably administered to a patient in the same manner, although the dosage is preferably two puffs applied to the affected areas once or twice daily.

In-vivo experimental results which demonstrate the efficacy of the foregoing compositions are set forth below. Specifically, the following working examples illustrate both the manner in which a representative sample of the compositions of the present invention have been used in human subjects suffering from at least one wart associated with infections of the human papillomavirus, and the experimental results obtained, which demonstrate the efficacy of the invention.

### Example 1

A juvenile male, specifically a 12-year old boy of Hispanic ancestry, was observed as having warts on three out of five fingers of the right hand. These warts were first filed in the manner set forth above for better contact with the medication, and they were then treated with a composition in gel form containing hydroxychloroquine as the only active ingredient (prepared in the manner set forth above for such compositions). This gel composition was applied to each of the warts, in an amount approximately equal to the surface area of each wart, once or twice a day for approximately one week, following which it was observed that all of the warts had disappeared completely, without leaving any visible scarring.

### Example 2

Another juvenile male, specifically a 15-year old boy also of Hispanic ancestry, presented with warts on his fingers. The same composition as in Example 1 was applied, in the same manner and with the same frequency as in Example 1, and after approximately one week of such treatments, similar results were observed; that is, all of the warts had disappeared completely, without leaving any visible scarring.

### Example 3

An adult male, specifically a 55-year old man of Hispanic ancestry, was observed to have genital warts, specifically, warts scattered about the skin of the scrotum. The same composition as in Example 1 was applied, in the same manner and with the same frequency as in Example 1, and after approximately one week of such treatments it was observed that 50% of the mass of the warts had disappeared. Thereafter, the patient switched to treatments with a composition in gel form containing chloroquine as the only active ingredient (prepared in the manner set forth above for such compositions), and after two days of similar twice-daily treatments with the latter composition, it was observed that the remaining 50% of the mass of the warts had disappeared.

### Example 4

An adult female, specifically a 50-year old black woman, and her male partner, a 24-year old man of mixed Hispanic and black ancestry, both of whom presented with genital warts. The female, who was also living with a human immunodeficiency virus (HIV) infection, reported warts on her labia majora, which were then treated with a composition in gel form containing chloroquine as the only active ingredient (prepared in the manner set forth above for such compositions). This gel composition was applied to each of the warts, in an amount approximately equal to the surface area of each wart, once or twice a day for approximately four weeks, after which the patient reported that she observed a great deal of improvement. Thereafter, the female patient switched to treatments with a composition in gel form containing chloroquine and amodiaquine as the active ingredients (prepared in the manner set forth above for such compositions), and after approximately two weeks of similar twice-daily treatments with the latter composition, she reported that the warts had disappeared.

The same treatment regime was used on her male partner, who presented with warts on his penis, and who, in addition, reported having been born with HIV. After three months it was observed that the warts had disappeared completely. It is believed that this patient required a longer duration of treatment to achieve a successful result due to his underlying HIV infection, which must have reduced the ability of his immune system to combat the human papillomavirus infection.

### Example 5

Another adult male, specifically a 36-year old man of Hispanic ancestry, was observed as having warts on one of his fingers. The same composition as in Example 1 was applied, in the same manner and with the same frequency as in Example 1, and after two days of such treatments, similar results were observed; that is, all of the warts had disappeared completely, without any visible scarring.

### Example 6

Another adult female, specifically a 45-year old woman of Hispanic ancestry, presented with warts on her fingers. These warts were treated with a composition in gel form containing chloroquine as the only active ingredient (prepared in the manner set forth above for such compositions), which was applied in the same manner as in Example 1. After three days of such treatments, the patient reported that all of the warts had disappeared completely, without any visible scarring.

### Example 7

Another juvenile male, specifically a 16-year old boy also of Hispanic ancestry, presented with two warts on his hand. After filing, a composition in gel form containing amodiaquine as the only active ingredient (prepared in the manner set forth above for such compositions) was applied to each wart, in the same manner and with the same frequency as in Example 1, and after approximately one week of such treatments, the warts were reduced in size by approximately 50%. Following a second week of similar treatments, both of the warts had disappeared completely.

### Example 8

Two juvenile females, specifically a 16-year old girl and her 14-year old sister, both also of Hispanic ancestry, each presented with warts on their feet—the 16-year old had two large warts on one foot, which made walking difficult for her without pain, while the 14-year old had one smaller wart on one of her feet. After filing, a composition in gel form containing the combination of chloroquine and hydroxychloroquine as the active ingredients (prepared in the manner set forth above for such compositions) was applied generously to all of the warts on the feet of both patients. After approximately three weeks of such treatments twice a day, all of the warts had disappeared completely from the feet of both patients.

### Example 9

An adult male, specifically a 50-year old man West African man from Senegal, presented with many different warts on his feet. After filing, a composition in gel form containing the combination of chloroquine, hydroxychloroquine and amodiaquine as the active ingredients (prepared in the manner set forth above for such compositions) was applied to the warts, as well as to the areas of his feet that were unaffected. After two weeks of such treatments twice a day, all of the warts had disappeared completely from both of his feet, allowing the patient to walk and wear shoes comfortably.

Based on the experimental results summarized above, it is believed that the present invention comprises treatment methods and compositions that can treat and remove the warts associated with infections of the human papillomavirus, and that can prevent the recurrence of such warts. It is also believed that the removal of such warts, particularly the rectal and genital warts and those found in the oral cavity associated with the sexually transmitted types of the human papillomavirus, will reduce the risk of spreading that virus, and will therefore subsequently reduce the morbidity and mortality rates associated with cervical, vaginal, vulvar, penile, anal, rectal and/or oropharyngeal cancers. It is further believed that the methods and compositions of the present invention can be used as a prophylactic treatment, to provide protection during sexual intercourse from infections of the human papillomavirus for individuals who have not yet been infected.

While there has been described what are at present considered to be the preferred embodiments of the present invention, it will be apparent to those skilled in the art that the embodiments described herein are by way of illustration and not of limitation. Various modifications of the disclosed embodiments, as well as alternative embodiments of the invention, will become apparent to persons skilled in the art upon reference to the description of the invention. Therefore, it is to be understood that various changes and modifications may be made in the embodiments disclosed herein without departing from the true spirit and scope of the present invention, as set forth in the appended claims, and it is contemplated that the appended claims will cover any such modifications or embodiments.

1. A method of treatment of an existing papillomavirus infection in a human in need thereof, comprising administering to said human a therapeutically effective amount of at least one compound selected from the group consisting of chloroquine, hydroxychloroquine, amodiaquine, and respective pharmaceutically acceptable salts thereof.

2. The method of treatment of claim 1 wherein said compound is optionally administered together with at least one pharmaceutically acceptable non-active ingredient.

3. The method of treatment of claim 1 or 2 wherein the papillomavirus infection is characterized by the presence of epithelial lesions.

4. The method of treatment of claim 3 wherein the epithelial lesions are selected from one or more of the group consisting of verrucae warts, flat warts, plantar warts and anogenital warts of the skin and mucosal surfaces.

5. A method of inhibiting or preventing the development of a papillomavirus infection in a human comprising administering to said human an antivirally effective amount of at least one compound selected from the group consisting of chloroquine, hydroxychloroquine, amodiaquine, and respective pharmaceutically acceptable salts thereof.

6. The method of treatment of claim 5 wherein said compound is optionally administered together with at least one pharmaceutically acceptable non-active ingredient.

7. A pharmaceutical composition for the treatment of a papillomavirus infection in a human host, comprising a therapeutically effective amount of at least one compound selected from the group consisting of chloroquine, hydroxychloroquine, amodiaquine, and respective pharmaceutically acceptable salts thereof.

8. The pharmaceutical composition of claim 7 further optionally comprising at least one pharmaceutically acceptable non-active ingredient.

9. The pharmaceutical composition of claim 7 or 8 wherein the papillomavirus infection is characterized by the presence of epithelial lesions.

10. The pharmaceutical composition of claim 9 wherein the epithelial lesions are selected from one or more of the group consisting of verrucae warts, flat warts, plantar warts and anogenital warts of the skin and mucosal surfaces.

11. A method of treating a viral disease selected from the group consisting of anogenital warts, verrucae warts, flat warts, plantar warts and papillomavirus in a human being, the method comprising the administration of the composition of claim 7 or 8 to a human being in need of such treatment.

12. A method for the treatment of virally-induced tumors in humans, wherein said tumors are associated with human papillomavirus and are selected from the group consisting of verrucae warts, flat warts, plantar warts and anogenital warts, the method comprising the application of the composition of claim 7 or 8 to a human in need of such treatment.
